(19) United States
(12) Patent Application Publication
(10) Pub. No.: US 2022/0115113 A1
(43) Pub. Date: Apr. 14, 2022

(54) Systems and Method for nutrition analytics for robotic kitchens

(71) Applicant: Kitchen Robotics Ltd, Modiin (IL)

(72) Inventors: Ofer ZINGER, Ramat HaSharon (IL); David BEN DAVID, Rehovot (IL)

(21) Appl. No.: 17/067,805

(22) Filed: Oct. 12, 2020

(51) Int. Cl. G16H 20/60

(52) U.S. Cl. CPC G16H 20/60

(57) ABSTRACT

The systems and methods of present invention analyzes a recipe's nutrition values, by using a data base to determine the nutrition values of the meal ingredients and displays dynamically in real time the nutrition analysis of consumer customizable recipes. The method and system involves calculation of nutrition value based on actual weight measurement of the ingredient, rather than the desired value required by the recipe.

### FIELD OF THE INVENTION

The present invention discusses systems and methods for undertaking nutritional analytics for robotic kitchens. Moreover, said invention allows precise estimation of nutrient values in a customized recipe thereby balancing customized nutritional requirements in an automatic environment.

### BACKGROUND OF THE INVENTION

Nutrition value and content of meals has become a necessity not only for healthy eating but as an effective tool for people with health conditions such as diabetes or food allergies, to name a few. Many seek healthy food so that they can achieve optimal nutrition and avoid developing co-morbidity conditions later in life. Forward-thinking foodservice providers have to take into account this major trend.

Presently, the caloric and nutritional content information for a prepared food is often minimal and sketchy. For example, when sugar is listed in the ingredient list, the consumer generally does receive any information about the source of the sugar, which can come from a variety of plants, such as sugarcane, beets, or corn, which will affect its nutritional content. Conversely, some nutritional information that is provided to consumers is so detailed, that they appear to a consumer, as useless information.

Hence, it is pertinent that accurate nutrition information that offers transparency apart from addressing an individual's specific dietary requirements is an essential pre-requisite. Furthermore, the information about nutritional substances in a typical recipe needs to be presented in a meaningful manner to enable satisfy the specific requirement of an individual. For example, consumers with a medical condition, such as diabetes, would want to track specific information regarding nutritional values associated with sugar and other nutrients in the foods and beverages they consume, and would benefit further from knowing changes in these values or having tools to quickly indicate or estimate these changes in a retrospective, current, or prospective fashion, and even tools to report these changes, or impressions of these changes, in a real-time fashion. Consumers would want to track medicaments for specific requirements, changes in their medicinal values, degradation, and for potential interactions with other medicaments and nutritional substances they are consuming or planning to consume.

As such there exist a requirement for a mechanism to share information and quality of the nutritional substances in a typical recipe, including calorific and nutritional, organoleptic, and aesthetic values. Consumers on being better informed about nutritional substances can select and consume in a better way thereby improving quality (taste, appearance, and caloric and nutritional content), efficiency, value and profit of the chosen recipes.

As consumers are demanding more information about what they consume, they are asking for a system that enables gauge nutritional content and more closely match good nutritional requirements that would meet their specific nutritional requirements.

Some granted inventions that discuss the relevance and plurality of perspective on measuring nutritional values are discussed:

U.S. Pat. No. 9,564,064B2 titled, “Conditioner with weight sensors for nutritional substances” disclose nutritional substance systems and methods for enabling the tracking and communication of changes in nutritional, organoleptic, and aesthetic values of nutritional substances, and further enabling the adaptive storage and adaptive conditioning of nutritional substances.

U.S. Pat. No. 5,412,560A titled, “Method for evaluating and analyzing food choices” discuss a process for evaluation of an individual's food choices based upon selected factors and dietary guidelines are disclosed. The DINE System provides a method and apparatus which allows an individual to determine how well or poorly the person is eating. The invention analyzes the food an individual eats and determines certain predictor and follower nutrients that will give rise to an assessment of how a person's diet matches with various dietary guidelines established by governmental and/or other entities. The invention provides the results of the analysis to the individual complete with messages regarding over or under consumption of key nutrients so that the individual can correct the diet thereby resulting in better eating habits. The invention also gives the individual a “score” by which the person can immediately assess how well he or she is doing with respect to the various guidelines. The higher the number the better the diet.

U.S. Pat. No. 5,478,989A titled, “Nutritional information system for shoppers” states a method for providing personalized nutrition information to an individual comprising the following steps: (a) inputting personal data relating to an individual; (b) inputting data identifying at least one food product which the individual intends to purchase or consume; (c) accessing prestored information relating to the at least one food product which the individual intends to purchase or consume; (d) generating and outputting information about the at least one food product which the individual intends to purchase or consume pertinent to the input personal data.

In none of these patented inventions, gauging the nutritional values of the ingredient in a typical recipe is discussed. It is thus necessary to develop a system for accurate gauging of the calorific/nutritional values of a specific recipe. Furthermore, as the meal nutrition value as is required in a specific manually prepared meal often varies and thus, the actual nutrition value varies, as well. As a result, the recipe derived meal nutrition value remains an estimate only, which might not be adequate for individuals with health conditions, planning their nutrition based on their meals nutrition values. Thus, a customized gauging of the nutritional values of a typical recipe is essential. The present invention works in that direction, more so, in a robotic environment, where automated recipes are developed that takes care of individualized dietary requirements besides accurately measuring the ingredients' calorific/nutritional values in a typical meal, by accurately analyzing their nutritional values.

### SUMMARY OF THE INVENTION

An aspect of the present invention is analyzing a recipe's nutrition values, where the fat, carbohydrates, and protein, among others, plus a host of other nutrients are calculated as per nutrition labels data base.

A further aspect of the invention is using a data base to determine the nutrition values of the meal ingredients. The data base might contain USDA National Nutrient Database, Canadian Nutrient File (CNF), and Public Health England's McCance and Widdowson's “The Composition of Foods”, as well as various commercial brands. The data base can be updated with the nutrition values of the specific brands of the used ingredient, thus resulting is accurate nutrition analysis of the recipe.

A still further aspect of the invention is a method of nutrition value calculation based on actual weight measurement of the ingredient, rather than the desired value required by the recipe.

Another aspect of the invention is nutrition value calculation based on actual weight measurement of the ingredient which is achieved by utilization of weight sensors.

A still further aspect of the invention is automatic adjustment or creation of a recipe, based on user input of dietary requirements or limitations. For example, an individual with specific health condition might require a recipe with limited amount of sodium.

A further aspect of the invention is providing a dynamic recipe nutrition profile to the user which enables the user to alter the recipe ingredients, and the user receives the recipe nutrition analysis in real time and the method fits the recipe to a predefined nutrition profile.

Another aspect of the invention is providing a method of nutrition analysis of consumer customizable recipe which displays the recipe nutrition values in real time, considering every consumer customization.

A still further aspect of the present invention is real time display of the recipe nutrition profile in consumer customizable recipe.

### DETAILED DESCRIPTION OF THE EMBODIMENTS

Various examples of the invention will now be described. The following description provides specific details for a thorough understanding and enabling description of these examples One skilled in the relevant art will understand, however, that the invention may be practiced without many of these details. Likewise, one skilled in the relevant art will also understand that the invention can include many other obvious features not described in detail herein.

In some embodiments the nutrition values of a recipe may be analyzed and calculated including the amount of fat, carbohydrates, and protein, among others, and a host of other nutrients as per nutrition label data base.

In some embodiments a data base may be used to determine the nutrition values of the meal ingredients. The data base may contain USDA National Nutrient Database, Canadian Nutrient File (CNF), and Public Health England's McCance and Widdowson's “The Composition of Foods”, as well as various commercial brands. The data base may be updated with the nutrition values of the specific brands of the used ingredient, thereby resulting is accurate nutrition analysis of the recipe.

For example: Alfredo sauce, commonly used in pasta dishes, contains certain amounts of sodium. A sauce supplied by Brand A might contain 390 mg of sodium per serving (¼ cup), vs. a sauce supplied by Brand B that might contain 370 mg of sodium per serving. A difference in this specific nutrition fact of the alfredo sauce has an influence on the nutrition analysis of the prepared meal. The method in the present invention enables adding an ingredient to a data base, such as an alfredo sauce, determining it by a specific brand. Thus, a meal including an ingredient from a specific brand, added to a database, is analyzed based on the specific nutrition values of the ingredient, resulting in accurate nutrition values of the meal.

In some embodiments, the nutrition value calculation of the recipe is based on actual weight measurement of the ingredient, rather than the desired value required by the recipe. Since robotic kitchens use various dispensing methods for ingredient collection processes, a specific ingredient may be dispensed or collected by controlling the actual ingredient weight vs. required by the recipe. The dispensing process might have an allowed error margin vs. the required weight.

The method described in this invention, receives the actual ingredient dispensed weight data and calculates the nutrition values accordingly. Robotic kitchens might include a mechanism measuring the weight of the dispensed ingredient. The mechanism may include:

receiving the actual dispensed ingredient weight from a weight sensor;

communicating the signal to weight processor through communication cable;

receiving the data from the weight processor by the system controller using communication protocol such as MUDBUS OR Ethernet;

receiving the weight data from the system main controller by the system software.

This mechanism may use a load cell as a weight sensor (such as a strain gauge load cell) or alternative weight or force sensors.

The method in this invention receives the actual weight value, returned by the weight measurement mechanism to the system controller, considering it in the meal nutrition analysis. Thus, the nutrition value of the prepared meal is derived from the actual dispensed weights of the recipe ingredients, rather than the estimated, desired values.

Additionally, the variation between the desired recipe weight and the actual dispensed ingredient weight is used to evaluate the dispensing parameters of the specific ingredient, such as dispensing duration and dispensing speed. For example, if the actual dispensed weight of an alfredo sauce is 78 grams, while the desired predefined weight required by the recipe is 70 grams, the nutrition values of the meal are derived from the actual weigh of 78 grams and the variation of 8 grams is considered as a correction feedback. Based on this feedback, the dispensing duration of the alfredo sauce can be reduced by a certain value to meet the desired weight required by the recipe.

Therefore, when the meal preparation is complete, the meal nutrition analysis, which is based on actual weight of the ingredients, is accurate, relative to the estimated nutrition analysis derived from the recipe.

In embodiments of the invention, the recipe customization inputs are received from an individual user through HMI, such as a touch screen. The input is received by the software, such as a cloud based software which transmits a command or set of commands to the system main controller, derived from the weight input. For example, if the user adds 10 grams of olive oil to a salad recipe, the system main controller will receive a set of commands, such as dispensing duration and dispensing speed, which will be transmitted to relevant dispensing units, such as the olive oil pump motor driver. The motor driver invokes the relevant motor, such as the olive oil dispensing pump motor, with the specified rotational speed for the specified duration, dispensing the required amount of the ingredient, for example 10 grams of olive oil.

In some embodiments the method incorporates customization of predefined recipe based on input of dietary requirements or limitations through automatic adjustment. For example, an individual with specific health condition might require a recipe with limited amount of sodium.

The method in the present invention reads as an input individual's dietary requirements, such as “low calorie meal”, “low sodium”, “low sugar” or “minimum 20 grams of protein”. The method analyzes the nutrition values of the recipes, and filters the recipe's dietary requirement inputs, receives the limitation as an input and calculates the required amounts in a specific recipe ingredient in order to meet the limitation.

In some embodiments, the method may present the individual user or consumer with the recipes complying the input requirements or alternatively guide the individual in creating a custom assembled meal by presenting the ingredients fulfilling the requirements. For example, in case an individual inputs “minimum 20 grains of protein” dietary requirement from a salad meal, the method will derive the protein values from the nutrition values of the salad ingredients and if the requirement is not met, suggest the individual to add a specific calculated amount of an ingredient from a list.

In some embodiments, the method of the present invention provides a dynamic recipe nutrition profile. Nutrition profiles serve as a simple tool assisting the consumers in choosing the recipes complying with their dietary requirements. Each profile uses different combination of factors like, fat, sodium, cholesterol, added sugars, vitamins, minerals, fiber and calories. The method in the present invention enables to alter the recipe ingredients, by receiving the recipe nutrition analysis in real time and fitting the recipe to a predefined nutrition profile.

In some embodiments the method provides for real time display of the recipe nutrition profile in consumer customizable recipe. Recipe nutrition profile, based on factors like fat, vitamins, calories etc., alters when consumer customizes the predefined recipe. Recipe customization, resulting in adding or removing ingredients or changing ingredients amount, might result in radical change of the recipe nutrition profile. Thus, originally presented nutrition profile, derived from the predefined recipe is no longer valid. The method in the present invention enables real time display of the recipe nutrition profile based on consumer customization choices. The nutrition profile of the recipe is displayed to the consumer, changing every time a customization is made.

For example, a nutrition profile of a meal might be, “low calorie meal”—a meal containing predefined number of calories. The recipe calories are derived from the nutrition values of the ingredients. However, in case the amount of dispensed olive oil, for example, exceeds the amount required by the recipe, the meal calorie value increases accordingly. Thus, the nutrition profile of the specific meal, characterizing it as a low-calorie meal, might no longer be valid. The method in the present invention applies a nutrition profile to a meal by considering the actual dispensed weight of the ingredients, for example, in case the amount of salt in a recipe exceeds the required value, the nutrition profile of the meal, originally defined as a “low sodium” meal might change to “high sodium meal”.

In some embodiments, the nutrition profile may be assigned to a recipe by characterizing the recipe nutrition values. For example, a recipe containing high amount of protein can be characterized as a “high protein” meal. However, the characteristics of the meal alter as the recipe is modified or customized. The customization might result in adding and removing an ingredient or changing the ingredients mount. For example, a “high protein” tuna salad might be customized, reducing the amount of tuna, thus, no longer meeting the “high protein” meal nutrition profile characteristics. The method derives the nutrition values of the reduced amount of tuna, calculates and displays in real time the nutrition value of the meal, characterizing the meal nutrition profile. Thus, reduction of the tuna from the “high protein” salad might result in a nutrition profile change to “low protein” meal.

In some embodiments the method also provides for nutrition analysis of consumer customizable recipe and displays the recipe nutrition values in real time, considering every consumer customization. Usually, predefined recipes are often being customized by the consumers, by adding and removing ingredients or changing the ingredient amount. In such a scenario, the initial estimated nutritional value of the recipe is no longer valid. Nutrition value of a meal is derived, among others, by the amount of its ingredients and their nutrition values, accordingly.

For example, in a predefined recipe, such as a salad, the amount of the ingredients is defined, thus the nutrition value of the meal can be derived. The customization of a predefined recipe is reflected in adding and removing ingredients or changing their amount. When adding olive oil for example, to a salad, the nutrition values of the salad, such as calories and fat change. The method derives the nutrition values of the added olive oil, calculating in real time the nutrition value of the salad, incrementally to the input amount of the olive oil. Thus, by displaying in real time the actual nutrition value of the meal, the system provides a real time feedback and real time analysis of the meal.

The present disclosed subject matter may be a system, a method, and/or a computer program product. The computer program product may include a computer readable storage medium (or media) having computer readable program instructions thereon for causing a processor to carry out aspects of the present disclosed subject matter. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of the present disclosed subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosed subject matter. Aspects of the present disclosed subject matter are described herein with reference to flowchart illustrations and/or block diagrams of methods, apparatus (systems), and computer program products according to embodiments of the disclosed subject matter. It will be understood that each block of the flowchart illustrations and/or block diagrams, and combinations of blocks in the flowchart illustrations and/or block diagrams, can be implemented by computer readable program instructions. These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosed subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the disclosed subject matter. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosed subject matter has been presented for purposes of illustration and description, but is not intended to be exhaustive or limited to the disclosed subject matter in the form disclosed. Many modifications and variations will be apparent to those of ordinary skill in the art without departing from the scope and spirit of the disclosed subject matter. The embodiment was chosen and described in order to best explain the principles of the disclosed subject matter and the practical application, and to enable others of ordinary skill in the art to understand the disclosed subject matter for various embodiments with various modifications as are suited to the particular use contemplated.

What is claimed is:

1. A system for analyzing nutritional value of a recipe for robotic kitchens, the system comprising:
a database with information on nutritional values of various brands;
a weight measurement sensor for detecting the weight of the ingredients of the recipe;
a controller configured to determine the weight of the ingredients based on the commands received from the weight measurement sensor; and
a display for outputting nutritional values of the recipe determined by the controller.

2. The system of claim 1, wherein the system further comprises an input panel configured to receive a consumer's input regarding the recipe ingredients.

3. The system of claim 1, wherein the database includes USDA National Nutrient Database, Canadian Nutrient File (CNF), and Public Health England's McCance and Widdowson's “The Composition of Foods”, and various commercial brands.

4. The system of claim 1, wherein the database is updated with nutritional values of ingredients based on specific brands.

5. The system of claim 1, wherein the nutritional value of the recipe is based on actual weight measurement of the ingredient.

6. A method for dispensing the actual ingredient weight required of a recipe for robotic kitchens, the method comprising:
receiving the actual dispensed ingredient weight from a weight sensor;
communicating the signal to weight processor through communication cable;
receiving the data from the weight processor by the system controller using communication protocol such as MUDBUS OR Ethernet.
receiving the weight data from the system main controller by the system software.

7. The method of claim 6, wherein the weight sensor is a strain gauge load cell.

8. The method of claim 6, wherein the weight sensors are force sensors.

9. The method of claim 6, wherein the dispensing parameters of the dispensing unit is determined by the variation between the desired recipe weight and the actual dispensed ingredient weight.

10. The method of claim 6, wherein the dispensing parameters are dispensing duration and dispensing speed.

11. The method of claim 6, wherein a nutrition profile is generated based on the actual dispensed weight of the ingredients.

12. The method of claim 6, wherein the actual dispensed ingredient weight determines the nutritional value of the recipe.

13. A system for analyzing nutritional value of a recipe based on the actual ingredient dispensed weight data, the system comprising:
a database with information on nutritional values of various brands;
a weight measurement sensor for detecting the weight of the ingredients of the recipe;
a controller configured to determine the weight of the ingredients based on the commands received from the weight measurement sensor;
a dispenser unit configured to receive data from the weight processor; and
a display for outputting nutritional values of the recipe determined by the controller.

14. The system of claim 12, wherein the system further comprises an input panel configured to receive a consumer's input regarding the recipe ingredients and dietary limitations.

15. The system of claim 12, wherein the weight measurement sensor is a strain gauge load cell.

16. The system of claim 12, wherein the weight measurement sensors are force sensors.

17. The system of claim 12, wherein the database includes USDA National Nutrient Database, Canadian Nutrient File (CNF), and Public Health England's McCance and Widdowson's “The Composition of Foods”, and various commercial brands.

18. The system of claim 12, wherein the database is updated with nutritional values of ingredients based on specific brands.

19. The system of claim 12, wherein the system incorporates customization of predefined recipes based on input of dietary limitations.

20. The system of claim 12, wherein the system guides the user with the recipes complying with the dietary limitations of the user.

21. The system of claim 12, wherein the system guides the user in developing a custom meal by presenting the ingredients fulfilling the user's requirement.

22. The system of claim 12, wherein a dynamic recipe nutrition profile is displayed realtime allowing the user to customize the ingredients of the recipe based on the user's requirement.

23. The system of claim 12, wherein nutritional analysis of consumer customizable recipe is displayed realtime, based on consumer customization.
